(19) United States
(12) Patent Application Publication
(10) Pub. No.: US 2005/0273080 A1
(43) Pub. Date: Dec. 8, 2005

(54) Methods and systems for providing an interface between an ambulatory medical device and a display device

(76) Inventor: Patrick J. Paul, Downingtown, PA (US)

Correspondence Address: RATNERPRESTIA, P O BOX 980, VALLEY FORGE, PA, 19482-0980, US

(21) Appl. No.: 11/132,834

(22) Filed: May 19, 2005

Related U.S. Application Data
(60) Provisional application No. 60/572,500, filed on May 20, 2004.

(51) Int. Cl. A61K 9/22

(52) U.S. Cl. 604/890.1; 128/920

(57) ABSTRACT

Systems and methods for providing an interface between an ambulatory medical device and a display device may comprise receiving a signal from the ambulatory medical device. The ambulatory medical device may be configured to produce the signal including information corresponding to data displayed on the ambulatory medical device. Systems and methods for providing an interface between an ambulatory medical device and a display device may also comprise transmitting the information corresponding to the data displayed on the ambulatory medical device to the display device. The display device may be configured to provide a larger view of the data displayed on the ambulatory medical device than is provided on the ambulatory medical device.

This application claims priority on provisional application Ser. No. 60/572,500, filed on May 20, 2004.

### FIELD OF THE INVENTION

The present invention generally relates to providing an interface. More particularly, the present invention relates to providing an interface between an ambulatory medical device and a display device. And even more particularly, the present invention relates to providing an interface between an ambulatory medical device and a display device wherein the ambulatory medical device comprises, for example, an ambulatory infusion pump or a glucometer, and the display device comprises, for example, a television.

### BACKGROUND INFORMATION

Devices, such as ambulatory external medical devices, may deliver material, such as insulin or medication, for example, through a tube and hollow needle (the infusion set) into a patient's body. Ambulatory external medical devices such as infusion pumps or glucometers are designed to be as small as possible, for example, to fit in clothing pockets, and more generally, small enough to be easily concealed. Manufacturers attempt to make displays, included on such devices, as large as possible, but are constrained by the device's small housing. Typically, however, the viewing area is often reported by many users to be small and difficult to read.

Adding to the readability difficulty of the ambulatory external medical device display is the limited capacity of the system's battery. Consequently, the chosen display technology has been a low-power-consumption LCD. LCD displays are notorious for providing poor contrast as well as limited viewing angles. These LCD displays are usually supplemented with a backlight technology to allow display viewing in low-ambient light conditions. To prevent excessive battery drain, however, the intensity of the backlight is often kept at a minimum. Thus, the LCD's readability is not improved significantly by the backlight. New display technology such as OLED (Organic Light Emitting Diode) address some of the contrast and directionality issues of LCD's, but the size of the fonts and/or icons is still too small for many users.

When the ambulatory external medical device comprises an insulin pump or a glucometer, for example, the need for excellent device readability may be further complicated by a user's diabetic eye disease. Diabetic eye disease is a group of eye disorders that afflicts people with diabetes. These eye diseases may include: i) diabetic retinopathy, which occurs when the tiny blood vessels that nourish the retina weaken, break down, or become blocked; ii) cataract, the clouding of the eye's natural lens; and iii) glaucoma, which develops when a fluid build-up within the eye damages the optic nerve.

In view of the foregoing, there is a need for systems and methods for allowing external ambulatory medical device users to read data from the device more easily.

### SUMMARY OF THE INVENTION

Consistent with exemplary embodiments of the present invention, systems and methods are disclosed for providing an interface between an ambulatory medical device and a display device.

In accordance with one exemplary embodiment of the present invention, a method for providing an interface between an ambulatory medical device and a display device comprises receiving a signal from the ambulatory medical device, the signal including information corresponding to data displayed on the ambulatory medical device, and transmitting the information corresponding to the data displayed on the ambulatory medical device to the display device, the display device configured to provide a larger view of the data displayed on the ambulatory medical device than is provided on the ambulatory medical device.

In accordance with another exemplary embodiment of the present invention, a system for providing an interface between an ambulatory medical device and a display device comprises a component configured for receiving a signal from the ambulatory medical device, the signal including information corresponding to data displayed on the ambulatory medical device, and a component configured for transmitting the information corresponding to the data displayed on the ambulatory medical device to the display device using a compatible video and audio format, the display device configured to provide a larger view of the data displayed on the ambulatory medical device than is provided on the ambulatory medical device.

In accordance with yet another exemplary embodiment of the present invention, a system for providing an interface between an ambulatory medical device and a display device comprises a memory storage for maintaining a database and a processing unit coupled to the memory storage, wherein the processing unit is operative to bidirectionally communicate data and command/control signals from and to the ambulatory medical device, the signal including information corresponding to data displayed on the ambulatory medical device and transmit the information corresponding to the data displayed on the ambulatory medical device to the display device using suitable audio and video formats, the display device configured to provide a larger view of the data displayed on the ambulatory medical device than is provided on the ambulatory medical device.

It is to be understood that both the foregoing general description and the following detailed description are exemplary and explanatory only, and should not be considered restrictive of the scope of the invention, as described and claimed. Further, features and/or variations may be provided in addition to those set forth herein. For example, embodiments of the invention may be directed to various combinations and sub-combinations of the features described in the detailed description.

### BRIEF DESCRIPTION OF THE DRAWINGS

Although the invention is illustrated and described herein with reference to specific embodiments, the invention is not intended to be limited to the details shown. Rather, various modifications may be made in the details within the scope and range of equivalents of the claims and without departing from the invention.

It is emphasized that, according to common practice, the various features of the drawings are not to scale. On the contrary, the dimensions of the various features are arbitrarily expanded or reduced for clarity. Included in the drawing are the following figures:

FIG. 1 is a block diagram of an exemplary interface system consistent with an exemplary embodiment of the present invention;

FIG. 2 is a flow chart of an exemplary method for providing an interface between an ambulatory medical device and a display device consistent with an exemplary embodiment of the present invention;

FIG. 3 is a flowchart of another exemplary method according to the present invention; and

FIG. 4 is a flowchart of yet another exemplary method according to the present invention.

### DETAILED DESCRIPTION

The following detailed description refers to the accompanying drawings. Wherever possible, the same reference numbers are used in the drawings and the following description to refer to the same or similar parts. While a presently preferred embodiment and features of the invention are described herein, modifications, adaptations and other implementations are possible, without departing from the spirit and scope of the invention. For example, substitutions, additions or modifications may be made to the components illustrated in the drawings, and the exemplary methods described herein may be modified by substituting, reordering, or adding steps to the disclosed methods. Accordingly, the following detailed description does not limit the invention. Instead, the proper scope of the invention is defined by the appended claims.

Systems and methods consistent with the invention provide an interface between an ambulatory medical device and a display device. For example, embodiments of the present invention include an ambulatory medical device containing a transmitter (wireless or not) that transmits within a certain distance of the device at least a portion of the information that appears on the device's screen. The ambulatory medical device's “screen information” can be simultaneously displayed on the ambulatory medical device screen and transmitted by the ambulatory medical device. The transmitted screen information is received by a receiver, encoded into a suitable format for a larger remote display, such as a television (TV) format (NTSC, PAL, SECAM, etc.). This signal may then be desirably combined with a cable or antenna TV signal in such a way that it occupies a TV channel with no other broadcast activity.

For simplicity, throughout the remainder of this description, the remote display device will be referred to as a TV. This is not intended to limit the present invention in any way, in that it is contemplated that other types of displays may be used, such as a monitor, or a computer display, for example.

In operation, anytime a user needs to use or program the ambulatory medical device, the user sets the TV to a predefined channel to display the device's “screen information.” Upon switching to this channel, the user may see on the TV screen information similar to that displayed on the ambulatory medical device's screen. In other words, after switching the TV to an “ambulatory device channel,” the user can operate the ambulatory medical device by actuating the device's keypad. However, the user no longer needs to look at the ambulatory medical device's screen, rather the user can look at the TV screen. Upon completing the ambulatory medical device's activation or programming, for example, the user can simply use the TV remote control to return to normal TV programming. The comparatively large bright TV screen make it easier for users to visualize the information shown. Furthermore, by using colors, it becomes easier to emphasize or de-emphasize certain types of information to refine the effectiveness of the user visual interface.

Moreover, in one exemplary embodiment, the system can, if it detects a signal emanating from the ambulatory medical device, automatically switch the TV to the ambulatory medical device channel. In addition, the system can automatically detect a channel without broadcast information (cable or antenna) and use the otherwise unoccupied channel (hereinafter referred top a “the ambulatory medical device channel”) to display the ambulatory medical device's information.

In one exemplary embodiment, the transmission by the ambulatory medical device can be used to turn-on the TV and switch it to the ambulatory medical device channel. In addition, the system can automatically switch the TV to the ambulatory medical device channel when the device transmits safety information such as alarms or warnings.

Furthermore, the display device can use the picture in picture (PIP) feature of more modern TVs to display usage/programming or alarms and warning situations. With respect to alarms and/or warnings, it is contemplated that these may be provided aurally, in addition to or as a substitute for visual indicators, in the event that the user is not presently viewing the TV screen when the condition that generates the alarm or warning occurs. Also, all the system's hardware and software can be fully integrated within the TV, such as in the form of an ASIC, for example. Moreover, the system can use any suitable communication protocol between the ambulatory medical device and the receiver such that any information is checked for data integrity and then confirmed back to the ambulatory medical device before the data is displayed.

Furthermore, the system can be programmed to recognize only specific device(s) and to ignore any transmission that does not incorporate the device's predetermined identification (ID). This ID may be incorporated in the transmission packet as a header for example or as part of selected data words sent by the ambulatory medical device. In another exemplary embodiment, the system can be adapted to recognize at least two devices at once or separately and display their respective information simultaneously or sequentially, as desired. For example, the system could be programmed to recognize both a glucometer and an insulin ambulatory medical pump and the user could visualize data from each device in two separate PIPs or in one combined image. Other devices that the system could recognize include glucose sensors (whether implanted or not). In addition, the system can contain a memory to store and retrieve information previously shown (screen history) so that the user or other caregiver can review historic information, as desired. The insulin pump and the glucometer can be, for example, two separate physical entities, working together as a system or working independently, but they can also be two distinct functions (pump and glucometer) working as a system within a shared common housing.

In addition, the system can use the TV's audio circuit to convey usage or programming information to the user or to get the user's attention when an alarm or a warning from the ambulatory medical device needs immediate attention. This feature can be used concurrently with TV screen information or in its place. Also, the user can elect to turn the ambulatory medical device's LCD screen off when the device is providing display data to a TV, for example, to extend battery life of the ambulatory medical device.

Moreover, the exemplary bidirectional communication protocol used between the ambulatory medical device and the receiver can use encryption techniques to prevent eavesdropping of the data or tampering with the transmission. Furthermore, the protocol can include well-known error checking routines or indictors, such a cyclic redundancy checking (CRC) for example, to identify any transmission corruption. To minimize the amount of data transmitted by the ambulatory medical device and thus improve the device's battery longevity, the system's communication protocol can incorporate well-known data compression techniques.

Further, if the system is subject to technical problems and is unable to properly display the ambulatory medical device information, the system can transmit a signal to the ambulatory medical device and cause the ambulatory medical device to record historic data to the ambulatory medical device's memory. Also, the system can execute self-diagnostics and place specific corresponding messages on the TV screen to provide the user with specific instructions.

In addition to immediate screen information, the ambulatory medical device may be adapted to transmit background information to the TV that can be used to supplement the simple duplication of the device screen to show, for example, a sliding bar indicating remaining ambulatory medical device battery energy or number of insulin units remaining in the cartridge. Thus, the TV screen may not only duplicate the ambulatory medical device screen, but it can also enhance it by complementing the information presented on the screen.

Furthermore, the system can support multiple languages. For example, the ambulatory medical device can download the necessary language strings and fonts for TV viewing into the database of the interface. With this feature, it is not necessary to support multiple languages in the interface device, rather the ambulatory medical device can download the necessary language information into the interface database.

The software necessary to operate the interface system can be downloaded from the ambulatory medical device. Accordingly, if the ambulatory medical device receives a software upgrade, it is possible to update the software associated with the interface system the next time the ambulatory medical device and the interface communicate.

An exemplary embodiment consistent with the invention may comprise an interface system. The system may provide an interface between an ambulatory medical device and a display device. The system may comprise a component configured to bidirectionally communicate with the ambulatory medical device. The ambulatory medical device may be configured to produce the signal, the signal including information corresponding to data displayed on the ambulatory medical device. Furthermore, the system may comprise a component configured for transmitting the information corresponding to the data displayed on the ambulatory medical device to the display device, using the suitable video and audio formats. The display device may be configured to provide a larger view of the data displayed on the ambulatory medical device than is provided on the ambulatory medical device.

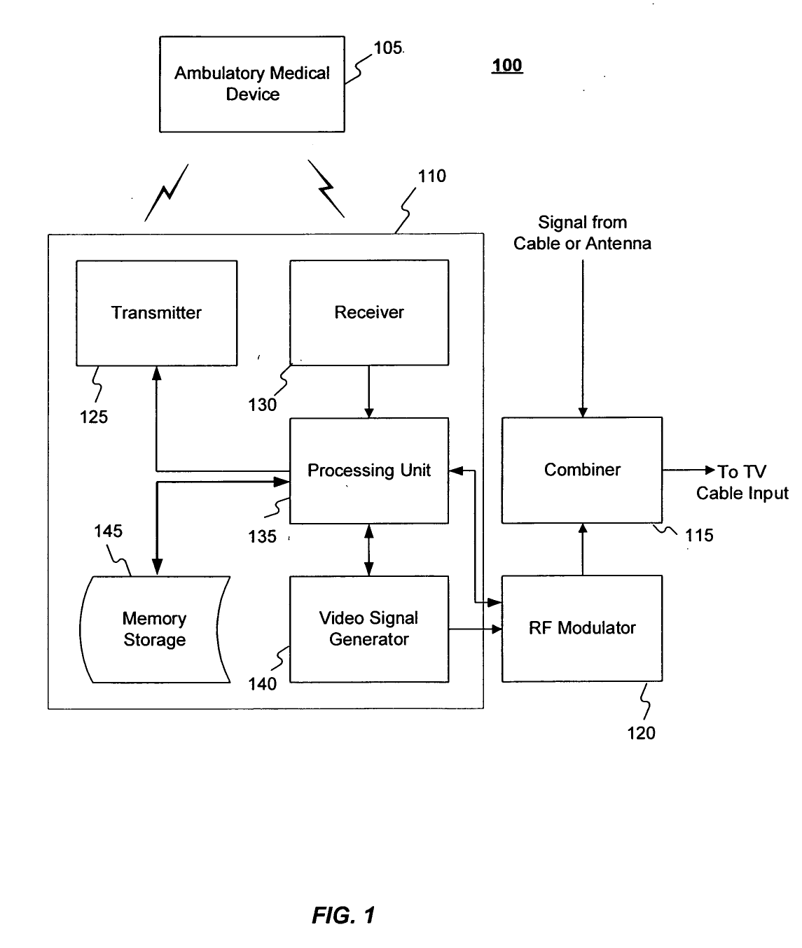

Consistent with an embodiment of the present invention, the aforementioned components may be implemented in an interface system, such as the exemplary interface system 100 of FIG. 1. Any suitable combination of hardware, software, and/or firmware may be used to implement the components such as a memory, a processing unit, and/or other components. By way of example, the memory, processing unit, or other components may be implemented with an interface device 110 in combination with system 100. The aforementioned system and device are exemplary, and other systems and devices may comprise the aforementioned memory, processing unit, or other components, consistent with embodiments of the present invention. As stated above, the system is not limited to a memory and a processing unit and may be implemented completely, for example, in hardware.

By way of a non-limiting example, FIG. 1 illustrates system 100 in which the features and principles of the present invention may be implemented. FIG. 1 illustrates an embodiment of the invention in which the display device may comprise a television receiver. While the display device may comprise a television receiver, it will be appreciated that the invention is not limited to a television receiver and other devices such as a monitor may be used as the display device. Furthermore, the invention is not limited to an ambulatory medical device and other devices may be used. As illustrated in the block diagram of FIG. 1, system 100 may include ambulatory medical device 105, interface device 110, combiner 115, and RF modulator 120. Interface device 110 may include transmitter 125, receiver 130, processing unit 135, video signal generator 140, and memory storage 145.

Ambulatory medical device 105 may comprise, but is not limited to an infusion device, a glucometer, or a device comprising an infusion device and a glucometer working in combination as separate physical devices, or as complementary functions within a shared common housing. A glucometer, for example, may be used as a remote controller for an infusion device. When ambulatory medical device 105 comprises a combination of devices, data or other information corresponding to both devices in the combination may be integrated and displayed. As described, ambulatory medical device 105 may comprise any device or combination of devices.

Transmitter 125 may transmit signals to ambulatory medical device 105 and receiver 130 may receive signals from ambulatory medical device 105. The signals may be transmitted and received utilizing a wire line communications system, a wireless communications system, or a combination of wire line and wireless. Wireless can be defined as radio transmission via the airwaves. However, it may be appreciated that various other communication and modulation techniques can be used to provide wireless transmission, including infrared, short-range RF, such as Bluetooth, cellular, microwave, acoustic, and spread spectrum radio, for example.

Processing unit 135 is adapted to process signals from receiver 130 to check data integrity and, for example, an ambulatory device ID of ambulatory medical device 105. Processing unit 135 may decode screen information and data to be displayed. Furthermore, processing unit 135 may direct control and command signals to transmitter 125 to confirm, for example, data with ambulatory medical device 105 and to signal particular status. Moreover, processing unit 135 may store information in memory 145.

Video signal generator 140 is adapted to convert the display information provided by processing unit 135 into a video signal. RF modulator 120 may generate a carrier frequency (channel frequency) corresponding to an available TV channel, for example. Furthermore, RF modulator 120 may modulate this carrier frequency with a video signal provided by video signal generator 140. In addition, RF modulator 120 may be controlled by processing unit 135 to select the carrier frequency (channel to use), for example. Combiner 115 may combine a signal provided by RF modulator 120 and the cable TV (or antenna), for example, into a single signal that may then be amplified (if necessary) and forwarded to an input of a television receiver (not shown). In addition, combiner 115 may provide a filter system that prevents the RF modulator signal from feeding-back into the antenna or cable TV feed. It may be noted that when a monitor is used as a display device instead of a TV, the video signal generator 140 may send its signal directly to the monitor, in which case RF modulator 120 and combiner 115 may not be required.

It should also be noted that while RF modulation/combination is mentioned above with respect to signal input to the display, the invention is not so limited. Indeed, other types of signal interface may be used, including (by non-limiting example) S-video, component video, Digital Video Interface (DVI), High Definition Multimedia Interface (HDMI), RGB, and various other IEEE and/or ANSI standards, either individually or in combination. Further, audio signals provided by the interface system to the display may be provided in one or more of a variety of formats. Non-limiting examples include, monaural, stereo, single channel of a stereo format, audio combined with a digital video signal (as in HDMI mentioned above), digital audio (wireless, coaxial and/or fiber optic).

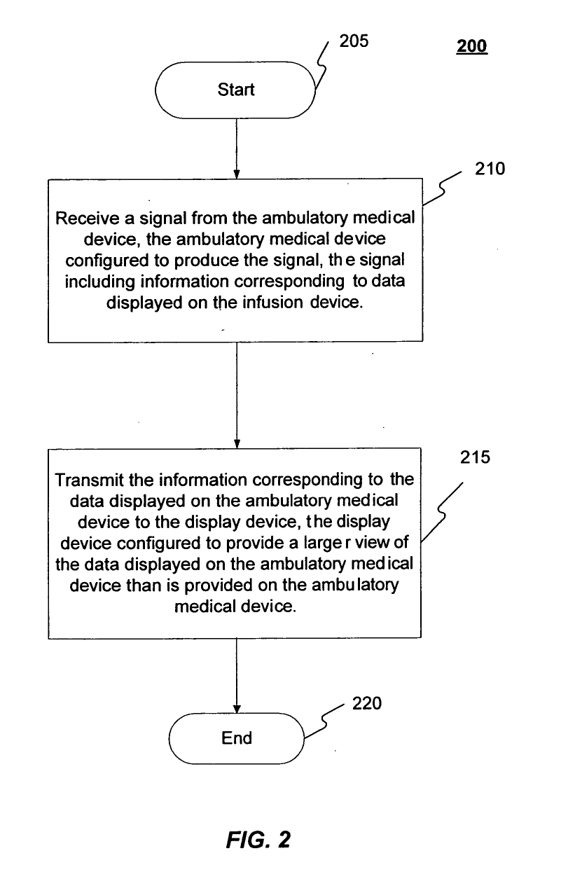

FIG. 2 is a flow chart setting forth the general stages involved in an exemplary method for providing an interface between an ambulatory medical device and a display device. Exemplary ways to implement the stages of method 200 will be described in greater detail below. Exemplary method 200 may begin at starting block 205 and proceed to stage 210 where interface device 110 may receive a signal from ambulatory medical device 105. Ambulatory medical device 105 may be configured to produce the signal. For example, the signal may include information corresponding to data displayed on ambulatory medical device 105.

From stage 210, where interface device 110 receives the signal from ambulatory medical device 105, exemplary method 200 may advance to stage 215 where interface device 110 may transmit the information corresponding to the data displayed on the ambulatory medical device to the display device. For example, the display device may be configured to provide a larger view of the data displayed on the ambulatory medical device than is provided on the ambulatory medical device. After interface device 110 transmits the information corresponding to the data displayed on the ambulatory medical device, exemplary method 200 ends at stage 220.

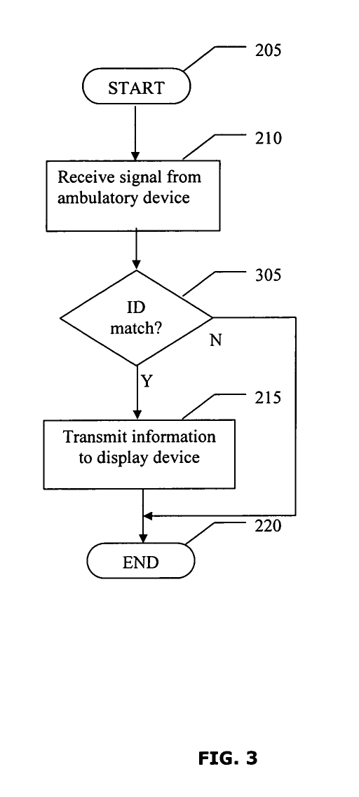

FIG. 3 is a flow chart of another exemplary method according to the present invention. The method of FIG. 3 is similar to that of FIG. 2 with the incorporation of additional steps implementing discrimination between signal emanating from recognized devices to those from unrecognized devices. In the exemplary method, this is achieved by Step 305 in which an ID associated with the signal received at Step 210 is checked to determine if it matches an expected ID. This may be accomplished for example by a look-up table, for example. If the ID matches Step 215 is entered, otherwise the process is terminated at Step 220.

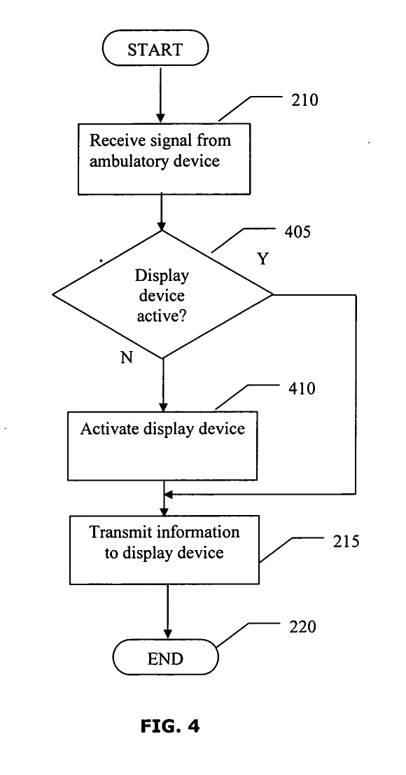

FIG. 4 is a flow chart of yet another exemplary method according to the present invention. The method of FIG. 4 is similar to that of FIG. 2 with the incorporation of additional steps for determining the state of the display device and then activating the display device if necessary before transmitting the information to the display device. In the exemplary method, this is achieved by Step 405 in which the status of the display device is determined. This determination may be achieved by various means, such as current draw of the display device, a feedback signal from the display device, for example. If the ID display device is not active Step 410 is entered and the display device is activated, otherwise the process skips to Step 215 and the information is transmitted to the display device.

While certain features and embodiments of the invention have been described, other embodiments of the invention will be apparent to those skilled in the art from consideration of the specification and practice of the embodiments of the invention disclosed herein. Furthermore, although embodiments of the present invention have been described as being associated with data stored in memory and other storage mediums, one skilled in the art will appreciate that these aspects can also be stored on or read from other types of computer-readable media, such as secondary storage devices, hard disks, floppy disks, a CD-ROM, a carrier wave from the Internet, or other forms of RAM or ROM. Further, the steps of the disclosed methods may be modified in any manner, including by reordering steps and/or inserting or deleting steps, without departing from the principles of the invention.

Although the methods of FIGS. 3 and 4 are shown as separate embodiments, the invention is not so limited in that the features of those embodiments may be combined as desired.

It is intended, therefore, that the specification be considered as exemplary only, with the true scope and spirit of the invention being indicated by the following claims and their full scope of equivalents.

1. A method for providing an interface between an ambulatory medical device and a display device, the method comprising:
receiving a signal from the ambulatory medical device, the signal including information corresponding to at least a portion of data displayed on the ambulatory medical device; and
transmitting the information corresponding to the data displayed on the ambulatory medical device to the display device, the display device configured to provide a larger view of the data displayed on the ambulatory medical device than is provided on the ambulatory medical device.

2. The method of claim 1, wherein the ambulatory medical device comprises one of an infusion device, a glucometer, and a device comprising a combination infusion device and glucometer operating together or not, as a system.

3. The method of claim 1, wherein the ambulatory medical device configured to produce the signal.

4. The method of claim 1, further comprising:
determining if the signal is from an approved ambulatory medical device; and
permitting the information to be transmitted to the display device based on the determination.

5. The method of claim 1, further comprising activating the display device based on the receiving of the signal from the ambulatory medical device.

6. The method of claim 1, further comprising displaying a first portion of the information in a first area of the display and a further portion of the information in another portion of the display.

7. The method of claim 1, further comprising providing at least one of a visual and/or aural warning from the display to a user based on a portion of the signal transmitted to the display.

8. The method of claim 1, aurally annunciating at least a portion of the information transmitted to the display device.

9. The method of claim 1, wherein the transmitting comprises a signal having an audio/video format.

10. A method for providing an interface between an infusion device, a glucometer, and a display device, the method comprising:
receiving a respective signal from at least one of the glucometer and the infusion device, both the glucometer and the infusion device being configured to produce the respective signal, the respective signal including information corresponding to at least a portion of data displayed on at least one of the glucometer and the infusion device; and
transmitting the information corresponding to the at least a portion of the data displayed on at least one of the glucometer and the infusion device, the display device configured to provide a larger view of the respective data displayed on at least one of the glucometer and the infusion device than is provided on at least one of the glucometer and the infusion device.

11. The method of claim 10, wherein the signal is received wirelessly.

12. The method of claim 10, wherein the information is transmitted wirelessly.

13. The method of claim 10, further comprising displaying information from the glucometer in a first area of the display and the information from the infusion device in another portion of the display.

14. The method of claim 13, further comprising displaying the information from the glucometer and the information from the infusion device sequentially on the display.

15. A system for providing an interface between an ambulatory medical device and a display device, the system comprising:
means for receiving a signal from the ambulatory medical device, the signal including information corresponding to at least a portion of data displayed on the ambulatory medical device; and
means for transmitting the information corresponding to the data displayed on the ambulatory medical device to the display device, the display device configured to provide a larger view of the data displayed on the ambulatory medical device than is provided on the ambulatory medical device.

16. The system according to claim 15, wherein the ambulatory medical device is configured to produce the signal.

17. The system according to claim 15, further comprising means for determining whether to transmit the information to the display device.

18. The system according to claim 15, further comprising means for activating the display device based on the received signal.

19. The system according to claim 15, further comprising means controlling the display device to display the information from the ambulatory medical device.

20. The system according to claim 15, wherein the ambulatory medical device comprises one of an infusion device, a glucometer, and a device comprising a combination infusion device and glucometer operating together or not, as a system.

21. The system according to claim 20, wherein the infusion device and the glucometer are contained within a common housing.

22. A system for providing an interface between an ambulatory medical device and a display device, the system comprising:
a memory for maintaining a database; and
a processing unit coupled to the memory, wherein the processing unit is operative to:
receive a signal from the ambulatory medical device, the signal including information corresponding to at least a portion of data displayed on the ambulatory medical device; and
transmit the information corresponding to the data displayed on the ambulatory medical device to the display device, the display device configured to provide a larger view of at least a portion of the data displayed on the ambulatory medical device than is provided on the ambulatory medical device.
